(19) United States
(12) Patent Application Publication
(10) Pub. No.: US 2003/0198952 A9
(43) Pub. Date: Oct. 23, 2003

(54) Probe bound substrate, process for manufacturing same, probe array, method of detecting target substance, method of specifying nucleotide sequence of single-stranded nucleic acid in sample, and quantitative determination of target substance in sample

(76) Inventors: Tadashi Okamoto, Yokohama-shi (JP); Nobuko Yamamoto, Isehara-shi (JP); Tomohiro Suzuki, Sagamihara-shi (JP)

Correspondence Address: FITZPATRICK CELLA HARPER & SCINTO, 30 ROCKEFELLER PLAZA, NEW YORK, NY, 10112, US

(21) Appl. No.: 09/764,420

(22) Filed: Jan. 19, 2001

(30) Foreign Application Priority Data
Jan. 28, 1999 (JP) 11-019915

(51) Int. Cl. C12Q 1/68; C12M 1/34

(52) U.S. Cl. 435/6; 435/287.2

(57) ABSTRACT

A probe bound substrate allowing us to quickly detect or quantify a target substance or sequence a target nucleic acid at a lower cost is provided. Specifically, there is provided a probe bound substrate in which a probe capable of specifically attaching to a target substance is bound at the first site on its surface, characterized in that a marker is bound at the second site where the first site may be specified.

### BACKGROUND OF THE INVENTION

1. Field of the Invention

This invention relates to a process for manufacturing a probe bound substrate, a probe array, a method of detecting a target substance and a method of specifying the nucleotide sequence of a single-stranded nucleic acid in a sample and a method of quantitatively determining a target substance in a sample.

2. Related Background Art

Recently, detection and quantification of a target substance using a solid-phase probe array has been intensely investigated and developed. For example, U.S. Pat. No. 5,445,936 has disclosed a solid-phase oligonucleotide array prepared using photolithography. Furthermore, PCT publication WO 95/25116 and U.S. Pat. No. 5,688,642 have disclosed a process for manufacturing a solid-phase DNA probe array using ink jet method. When detecting or quantifying a target substance using a probe array, it is important to know which probe has reacted with the target substance, among the probes in the array.

We have intensely investigated a technique for, e.g., detecting and/or quantifying a target substance using a probe array prepared by a variety of processes, and have found an additional technical problem as described below which has not been understood. Specifically, when a probe array is prepared by photolithography, it is relatively easier to set each probe in a position corresponding to a particular place on a substrate. However, when preparing a solid-phase probe array by ink jet technique, it may be difficult to set each probe in a position corresponding to a particular place on a substrate due to variation in a device used (a mask aligner is used in photolithography), compared to the above process using photolithography. Specifically, when detecting and/or quantifying a target substance by a fluorescent technique, relative positions for individual probes on the substrate can be determined if all or an adequate number of the sites in which a probe has been bound emit fluorolescence and thus positions of the individual sites can be relatively easily specified on the substrate. It may be, however, frequent that fluorescence is observed only from a particular site. In such a case, it is difficult to determine relative positions for individual probes and thus the probes permitting the sites to emit fluorescence may not be specified. Such a problem may be to some extent solved by forming a matrix pattern on a substrate in advance, but the use of such a substrate may cancel out the advantage of the process for manufacturing a probe array by ink jet technique that the probe array may be formed at a lower cost.

### SUMMARY OF THE INVENTION

In view of such a newly recognized technical problem, an objective of this invention is to provide a probe bound substrate allowing us to quickly detect or quantify a target substance or sequence a target nucleic acid at a lower cost and a manufacturing process therefor.

Another objective of this invention is to provide a probe array allowing us to quickly detect or quantify a target substance or sequence a target nucleic acid at a lower cost.

Further objective of this invention is to provide a method of quickly detecting the presence of a target substance in a sample at a lower cost.

Further objective of this invention is to provide a method of quickly sequencing a single-stranded nucleic acid in a sample at a lower cost.

Further objective of this invention is to provide a method of quantifying a target substance in a sample at a lower cost.

According to one aspect of the present invention, there in provided a probe bound substrate on which a probe capable of specifically attaching to a target substance is bound at the first site on a surface of the substrate, characterized in that a marker is bound at the second site where the first site can be specified.

According to an other aspect of the present invention, there is provided a probe bound substrate comprising the steps of applying a solution containing a probe capable of specifically making a bond with a target substance and having a second functional group capable of making a bond with a first functional group attached to the surface of a substrate, to a first site of a surface of a substrate and binding the probe to the substrate at the first site of a substrate surface, further comprising the step of applying a solution containing a marker having a third functional group capable of directly or indirectly making a bond with the first functional group to a second site of the substrate surface binding the maker to the second position of the substrate surface and wherein the first site can be specified from the second site.

According to a further aspect of the present invention, there is provided a probe array comprising spots for mutually independent probes at multiple sites on a substrate surface wherein a marker is present on the substrate surface such that the positions of the spots can be specified.

According to still another aspect of the present invention, there is provided a method of detecting a target substance comprising the steps of contacting a sample with each spot in a probe array on a substrate, having probes capable of specifically making a bond with a target substance possibly contained in the sample as a plurality of mutually independent sopts, wherein a marker is present on a substrate surface such that the positions of the spots can be specified, and detecting the presence of a reaction product of the probe with the target substance in any spot to detect the presence of the target substance in the sample, further comprising the step of specifying the positions of the spots where the reaction product is present on the basis of the positions of the marker on the substrate surface when the presence of the reaction product is detected.

According to still another aspect of the present invention, there is provided a method of sequencing a single-stranded nucleic acid in a sample comprising the steps of: contacting a sample with each spot in a probe array having probes having a complementary sequence to each of expected multiple sequences in the single-stranded nucleic acid as a plurality of mutually independent spots, wherein a marker is present on the substrate surface such that the positions of the spots can be specified, and specifying the positions of the spots where a reaction product of the probe with the target substance has been formed on the basis of the positions of the marker on the substrate.

According to still another aspect of the present invention, there is provided a method of quantifying a target substance wherein the quantity of fluorescence generated from a marker is used as a standard fluorescence quantity in a procedure where a probe array having mutually independent probe spots at multiple positions on a substrate surface in which a marker is present on the substrate surface such that the positions of the spots can be specified is used for detecting and quantifying the target substance capable of specifically making a bond with the probes by a fluorescent technique.

According to the present invention as described above, the positions of spots in each probe can be quickly and accurately specified even when probes are densely disposed as spots on a flat substrate without, e.g., wells using ink jet technique.

### BRIEF DESCRIPTION OF THE DRAWINGS

FIG. 1 shows the target DNA and the probe sequences in Example 2 and arrangement thereof on the array.

FIG. 2 shows a dot pattern of each DNA probe or marker in Example 2.

### DETAILED DESCRIPTION OF THE PREFERRED EMBODIMENTS

This invention will be detailed with reference to the drawings.

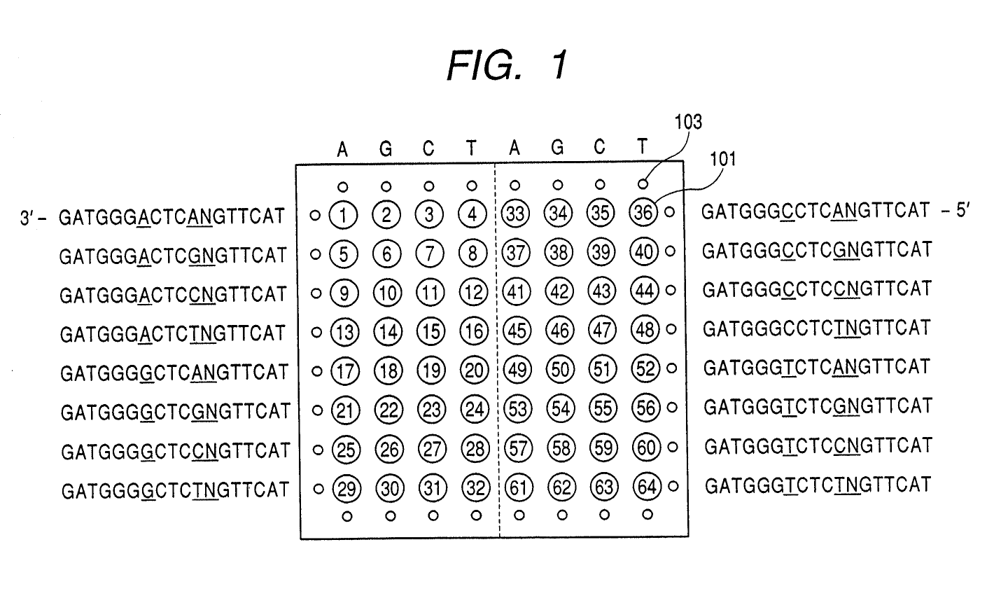

FIG. 1 is a plan view of a probe array where multiple probes with mutually different sequences are bound to a substrate surface as spots. In this figure, 101 is a spot for a probe and 103 is a spot for a marker, which is disposed at a position from which the position of the spot 101 can be specified. Specifically, the marker spots are disposed at the positions corresponding to each raw and each column of the probe spots 101 in a matrix, whereby the positions of the spots 101 can be specified. In this figure, a number in a probe spot is given for convenience of description in Examples later.

The spots for a marker 103 may be formed on the substrate, for example, by applying a solution containing the marker to the substrate by an appropriate method such as ink jet technique. The probe spots 101 may be also formed by applying a solution containing a probe by ink jet technique. The marker spots 103 may be preferably formed simultaneously with formation of the probe spots 101 in one step of ink jet application for avoiding misalignment between the rows or the columns of the probe spots and the marker spots 103.

### Marker

Any substance may be used as a marker as long as it can provide detectable information, e.g., fluorescence, in the state that it is present on a substrate. For example, a dye may be preferably used because it may provide a marker spot detectable by an optical microscope. Furthermore, a target substance is frequently detected in a solid-phase probe array using a fluorescent-labeling material. In this sense, it is convenient that the marker is a fluorescent material because the marker may be detected simultaneously with a target substance using a single device. A fluorescent dye is generally used as a fluorescent material. For example, a fluorescent dye having the same structure as a labeling material used in detecting a target substance may be advantageously used, e.g., for permitting them to be simultaneously observed using a fluorescence microscope. On the other hand, different dyes may be used to prevent the marker from disturbing detection of the target substance. Specific marker compounds which may be used in this invention include fluoresceine, rhodamine B, tetramethylrhodamine, rhodamine X, Texas Red and CY5.

A marker may be simply attached to a substrate. However, taking into consideration the case that a probe array is washed after reaction with a target substance, it is preferable that the marker is chemically bound to the substrate to prevent the marker from being removed by washing etc. There are no restrictions to a method for binding the marker to the substrate, and any appropriate method may be employed. In a preferable aspect of this invention, mutually reactive functional groups are introduced in the marker and the substrate surface, respectively, for forming a chemical bond between the substrate and the marker immediately after applying the solution containing the marker to the substrate when applying the marker to the substrate by ink jet technique as described above. Examples of a combination of functional groups in a substrate and in a marker are as follows:

(1) maleimide as a functional group on the substrate surface and thiol as a functional group in the marker;

(2) thiol as a functional group on the substrate surface and maleimide as a functional group in the marker;

(3) succinimide as a functional group on the substrate surface and amino as a functional group in the marker;

(4) amino as a functional group on the substrate surface and succinimide as a functional group in the marker;

(5) isocyanate as a functional group on the substrate surface and amino as a functional group in the marker;

(6) amino as a functional group on the substrate surface and isocyanate as a functional group in the marker;

(7) chloride as a functional group on the substrate surface and hydroxyl as a functional group in the marker;

(8) epoxy as a functional group on the substrate surface and amino as a functional group in the marker;

(9) carboxyl as a functional group on the substrate surface and hydroxyl as a functional group in the marker; and

(10) hydroxyl as a functional group on the substrate surface and carboxyl as a functional group in the marker.

A marker may be appropriately selected from these combinations, considering factors such as the structure of the specific compound used as a marker and the substrate material.

### Solid-Phase Substrate

There are no restrictions to a substrate material as long as it can bind the probe and the marker and it does not disturb detection of a target substance; for example, a glass substrate may be used. In addition, it may be a silicon, metal or resin substrate which may be optionally subject to surface processing. When using a glass substrate as a substrate, a variety of procedures for washing, surface processing and so on are well-known, and the material is suitable because of advantages that the substrate itself is readily available, etc. A functional group may be introduced on the surface of the glass substrate by, for example, introducing an appropriate group such as hydroxyl and carboxyl by any of various surface processing methods and these functional groups may be used as they are. Alternatively, a glass substrate may be treated with a silane coupling agent having a variety of functional groups and the functional groups may be utilized. Functional groups in commercially available silane coupling agents include thiol (SH), amino, isocyanate, chloride and epoxy, from which a functional group capable of making a bond with the functional group in the silane coupling agent may be appropriately selected to be used as a functional group involved in binding of the probe or the marker to the substrate. Processing with a silane coupling agent is well-known and thus not herein described in detail. Specifically, silane coupling agents having the above functional groups which may be used are available from Shin-Etsu Chemical Co. Ltd. and Nippon Uniker Co. Ltd.

### Composition of an Ink Jet Solution

Various methods described above may be employed for applying a marker to the above substrate, but ink jet technique whereby a fine droplet with a volume of several pl to several ten nl may be discharged is suitable. Practically available ink jet techniques to date include piezo jet technique using a piezo device and thermal jet technique using a thermal device. Either of these may be employed in this invention. When applying a marker to the above substrate surface by ink jet technique, it is preferable to adjust a solution composition such that a droplet may not be unnecessarily spread on the substrate for remaining in a given position. Furthermore, the solution composition is preferably that which does not adversely affect the intended performance of the marker or reduce reactivity of the functional group introduced in the marker with the functional group in the substrate surface.

When using ink jet technique, the substrate is preferably stored in a reaction vessel such as a moisture-keeping vessel during the reaction for preventing the droplets applied on the substrate surface from being evaporated and dried due to their fineness. Alternatively, it may be effective to add a moisturizing agent in the solution to be applied. Particularly, thermal jet technique is associated with temperature rising during discharge and therefore, it is important to add a moisturizing agent and a surface-tension adjusting agent. Such a marker or a solvent for applying a probe to the substrate surface may be suitably a solution containing 5 to 10 wt % of urea, 5 to 10 wt % of glycerol, 5 to 10 wt % of thioglycol and 1 wt % of an acetylene alcohol. The acetylene alcohol has the structure represented by general formula I

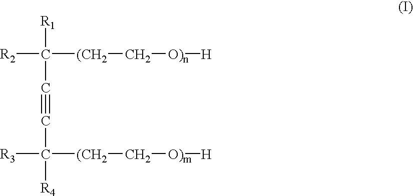

wherein R1, R2, R3 and R4 independently represent alkyl, specifically straight or branched alkyl with 1 to 4 carbon atoms; m and n independently represent an integer provided that m or n is zero when m=n=0 or 1≦m+n≦30 and m+n=1.

### Probe

A probe used in this invention is specifically bound to a target substance and it may, if necessary, contain a label for detecting that it has been bound to the target substance. A typical material used as a probe may be a single-stranded nucleic acid, including a single-stranded DNA, a single-stranded RNA and a single stranded PNA (peptide nucleic acid). Such a probe may be selected from known materials as appropriate depending on the type of the target substance. This invention may encompass a system where mutually reactive functional groups are introduced in a probe and a substrate to ensure binding of the probe to the substrate as described above for a marker. Examples of a combination of functional groups which may be introduced in a probe and a substrate include amino (probe side)—epoxy (substrate side) and thiol (probe side)—maleimide (substrate side).

### SH and Maleimide Groups

A preferable combination may be maleimide and thiol (—SH). Specifically, a thiol group (—SH) is bound to the terminal of a nucleic probe while a solid-phase surface is processed to have a maleimide group. Thus, when applying the probe to the solid-phase surface, the thiol group in the nucleic acid probe is reacted with the maleimide group on the solid-phase surface to immobilize the nucleic acid, resulting in forming a spot of the nucleic acid probe on a given position. Particularly, a nucleic acid probe solution may form a considerably fine spot on a solid-phase surface by applying the solution of a nucleic acid probe having a thiol group in its terminal with the above composition to the solid-phase surface on which a maleimide group has been introduced, using a bubble jet head. Thus, a fine spot of the nucleic acid probe may be formed at a given position on the solid-phase surface. In this case, it is not necessary to, for example, form in advance wells consisting of hydrophilic and hydrophobic matrices on the solid-phase surface for preventing spots from being combined.

For example, an 8 μM solution of a nucleic acid probe with a base length of 18-mer whose viscosity and surface tension were adjusted within the above range was discharged from a nozzle of a bubble jet printer (trade name: BJC 620; Canon Inc.) modified to be able to make printing on a flat plate while setting a distance between the solid and the nozzle of the bubble jet head of about 1.2 to 1.5 mm and a discharge amount of about 24 picoliters. As a result, a spot with a diameter of about 70 to 100 μm could be formed on the solid with no visible spots due to splash when the solution reached the solid-phase surface (hereinafter, referred to as a “satellite spot”). The reaction of the maleimide group on the solid phase with the SH group at the terminal of the nucleic acid probe may be completed in about 30 min at room temperature (25° C.) depending on the conditions of the liquid discharged. The time is shorter than that taken when using a piezo jet head for discharging the liquid. Although the reason is unknown, it might be because in bubble jet technique, the liquid containing a nucleic acid probe is warmed in the head in principle so that the reaction between the maleimide and the thiol groups becomes more efficient to reduce a reaction time.

When using the combination of maleimide and thiol, the solution containing the nucleic acid probe preferably contain thiodiglycol. A thiol group may be dimerized by forming a disulfide bond (—S-S—) under a neutral or weakly alkaline condition. Addition of thiodiglycol may, however, prevent reduction in reactivity of the thiol group with the maleimide group due to dimer formation.

A maleimide group may be introduced on a solid-phase surface by a variety of methods; for example, an aminosilane coupling agent may be reacted with a glass substrate and the amino group may be then reacted with a reagent containing N-(6-maleimidocaproyloxy)succinimide represented by the following structural formula (EMCS reagent; Dojin Co. Ltd.).

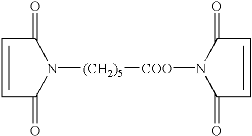

A nucleic acid probe having a thiol group may be synthesized by using 5′-Thiol-Modifier C6 (Glen Research Inc.) when automatically synthesizing a DNA using an automatic DNA synthesizer and usually purified by high performance liquid chromatography after deprotection.

### Amino and Epoxy Groups

In addition to the above combination of thiol and maleimide groups, a combination of functional groups used in immobilization may be, for example, a combination of an epoxy group (an a solid phase) and an amino group (nucleic acid probe terminal). An epoxy group may be introduced on the solid-phase surface by, for example, applying polyglycidyl methacrylate having an epoxy group to a resin solid-phase surface or a silane coupling agent having an epoxy group to a glass solid-phase surface for reaction with the glass.

Functional groups mutually reactive to form a covalent bond may be introduced on a solid-phase surface and at a terminal of a single-stranded nucleic acid probe to form a stronger bond between the nucleic acid probe and the solid phase. The nucleic acid probe can be always bound to the solid phase at its terminal, so that the nucleic acid probe may be in a homogeneous state at all spots. Thus, the conditions may be uniform in hybridization between the nucleic acid probe and a target nucleic acid to allow us to more accurately detect the target nucleic acid or more precisely specify its sequence. Furthermore, covalently bindinig the nucleic acid probe having a functional group at its terminal to the solid phase may permit a probe array to be quantitatively prepared without difference in a binding amount of the probe DNA due to variation in a sequence or length, in contrast to a nucleic acid probe immobilized on a solid by non-convalent bond such as an electrostatic bond. Additionally, all of the sequence in the nucleic acid may contribute to the hybridization reaction to significantly improve an efficiency of the hybridization reaction. A linker such as an alkylene group with 1 to 7 carbon atoms may be introduced between a part involved in hybridization between a single-stranded nucleic acid probe and a target nucleic acid and a functional group involved in a reaction with a solid phase. Thus, a given distance may be provided between the solid-phase surface and the nucleic acid probe when binding the solid phase with the nucleic acid probe and may further improve an efficiency of the reaction between the nucleic acid probe and the target nucleic acid.

### Linker

An appropriate linker may be inserted between a substrate and a probe in order to various purposes such as more effective detection of a target substance, variation in a distance between the substrate and a probe and making various functional groups available for the substrate and the probe, where the linker is, of course, inserted between the substrate and the marker. Typical examples of a system to which the method is applicable include that where a functional group to be bound to a linker in a silane coupling agent is amino, functional groups at the first and the second terminals in the linker are succinimide and maleimide, respectively, and a functional group in a marker is thiol or that where a functional group to be bound to a linker in a silane coupling agent is thiol, functional groups at the first and the second terminals in the linker are maleimide and succinimide, respectively, and a functional group in a marker is amino. In these systems, bond-forming reactions, of course, occur between the functional groups of the silane coupling agent and of the first terminal in the linker and between the functional groups of the marker and of the second terminal in the linker.

A linker which may be used in the above two systems may be a substance comprising a succinimide group capable of making a bond with an amino group at the one terminal and a maleimide group capable of making a bond with a thiol group at the other terminal. Various types of such substances which can be used in this invention are commercially available from Sigma Aldrich Japan and Dojindo Laboratories. Among these commercially available substances, since both succinimide and maleimide groups are readily hydrolyzable, substances exhibiting a degradation rate as low as possible are desirable; preferably N-(6-maleimidocaproyloxy)succinimide (EMCS; Compound II).

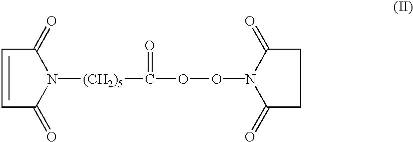

Although a maleimide group capable of selectively reacting with a thiol group is herein given as an exemplary functional group capable of making a bond with a solid-phase substrate probe or a marker, there are no commercially available fluorescent dyes having a thiol group when using a fluorescent dye. In such a case, a commercially available fluorescent dye may be appropriately chemically modified. For example, various fluorescent dyes having an amino group are known and commercially available. Thus, N-succinimidyl-3-(2-pyridyldithio)propionate (SPNP; Compound III) may be bound to the amino group and then a disulfide (—SS—) bond formed may be cleaved with, for example, dithiothreitol to give a thiol which can be used. An example of a fluorescent dye having an amino group is 5-(and 6-)[{N-(5-aminopentyl)amino}carbonyl]tetramethylrhodamine (tetramethylrhodamine cadaverine; Compound IV).

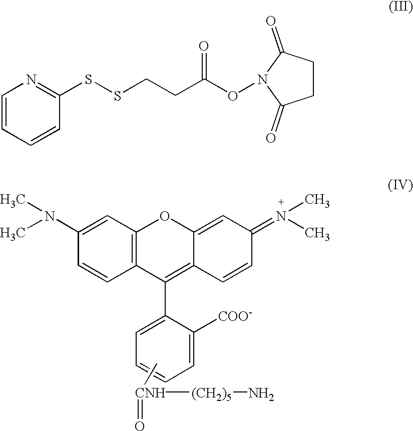

A frequently used procedure for detecting, in particular quantifying a target substance using a probe array is generally that a control region is formed in the array and the area is treated with a labeling model target with a known concentration to provide a signal from the labeling material, which is used to quantify a target substance with an unknown concentration. A region marked according to a marking method of this invention may be used for a similar purpose or as a standard for an absolute signal intensity.

### EXAMPLES

This invention will be specifically described with reference to Examples.

### Example 1

Preparation of a Marker Having a Thiol Group

In a reaction vessel was placed 1 mg of 5-(and 6-) [{N-(5-aminopentyl)amino}carbonyl]tetramethylrhodamine (tetramethylrhodamine cadaverine; Compound IV, Funakoshi Yakuhin Co. Ltd., 1.95 μmol) and it was dissolved in 0.5 mL of ethanol. To the solution was added a solution of 1.2 mg of N-succinimidyl-3-(2-pyridyldithio)propionate (SPDP; Compound III, Dojindo Laboratories, 3.85 μmol) in 0.5 mL of ethanol, and the mixture was reacted with stirring at room temperature for two hours. After confirming completion of the reaction by a thin layer chromatography, a desired compound (Compound V) was purified using a silica gel chromatography solid extracting tube (SUPELCO LC-SI; Sigma Aldrich Japan) and used in the next reaction without further purification.

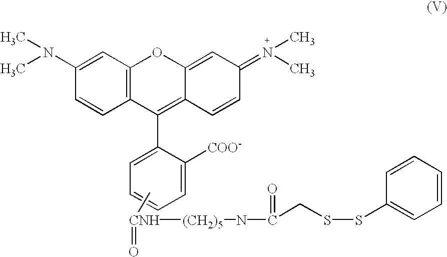

The whole amount of Compound V was dissolved in 0.5 mL of ethanol and to the mixture was added a solution of 2 mg of dithiothreitol (excess) in 0.5 mL of ethanol. The mixture was reacted by stirring at room temperature for two hours. After confirming completion of the reaction by a thin layer chromatography, a desired compound (Compound VI) was purified using the above silica gel chromatography solid extracting tube. Whether the synthesis of the compound VI was successful or not was determined by the presence of the attachment to the solid-phase substrate in the Example 2 because of its expensive material and a small quantities of both of yield and necessity.

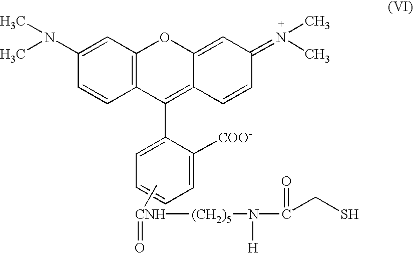

### Example 2

Attachment of Compound VI on a Solid Substrate

A fused quartz substrate with a size of 25.4 mm×25.4 mm×0.5t was subject to ultrasonic cleaning for 20 min in a 1% detergent exclusively for ultrasonic cleaning GP-II (Branson) and then in tap water and finally washed with running water as appropriate. Then, it was immersed in 1 N NaCl at 80° C. for 20 min, washed with running water (tap water), cleaned by ultrasonic in extrapure water, and washed with running water (extrapure water).

A 1% aqueous solution of an aminosilane coupling agent (KBM-603; Compound VII, Shin-Etsu Chemical Co. Ltd.) purified by vacuum distillation was stirred for one hour at room temperature to hydrolyze its methoxy moiety. This procedure is recommended by the manufacturer and is common for dealing with a silane coupling agent. Then, the above substrate immediately after washing was immersed in the above aqueous solution of silane coupling agent for one hour, washed with running water (extrapure water), dried by nitrogen gas blowing and fixed by heating in an oven at 120° C. for one hour.

(CH₃0)₃Si(CH₂)₃NH(CH₂)₂NH₂ (VII)

After cooling, the substrate was immersed in a 0.3% solution of N-(6-maleimidocaproxy)succinimide (EMCS; Compound II) of ethanol and dimethylsulfoxide (1:1) at room temperature for two hours for reaction, washed with a mixture of ethanol: dimethylsulfoxide=1:1 once and with ethanol three times and dried by nitrogen gas blowing.

Compound VI in Example 1 was dissolved in a solvent for discharge from a thermal jet printer, i.e, an aqueous solution of 7.5 wt % of glycerol, 7.5 wt % of urea and 1 wt % of thiodiglycol 7 (Renol EH; Kawaken Fine Chemical Co. Ltd.) to an absorbance of 1.0. Two milliliters of the solution was filled in an ink tank in a thermal jet printer (BJC-600J; Canon Inc.) and was discharged on the above substrate. The BJC-600J used was modified so as to perform discharge on, e.g, a glass substrate. According to the specifications of the device, a size of one droplet discharged is 24 pl. Under these conditions, a dot diameter occupied by one droplet is 70 to 100 μm. A discharge density is 120 dpi (dot/inch) and the number of discharged droplets is 50×50=2500. The substrate on which the solution of Compound VI was discharged was reacted in a moisturizing chamber with a humidity of 100% at room temperature for one hour and then washed in running water (extrapure water) for about 30 sec.

Then, for matching the conditions with those in a DNA array described later, the above substrate was immersed in a 50 mM phosphate buffer (pH=7.0, containing 0.1 M NaCl) containing 2% BSA (bovine serum albumin; Sigma Aldrich Japan), washed with the above buffer as appropriate, placed on a slide glass as it was and covered with a cover glass for observing fluorescence. A fluorescence microscope used was ECLIPSE E800 (Nikon Co. Ltd.) equipped with a 20× object lens (Planapochromate) and a fluorescence filter (Y-2E/C). An image was taken using a CCD camera (C2400-87; Hamamatsu Photonics Co. Ltd.) equipped with an image intensifier and an image processor (Argus 50; Hamamatsu Photonics Co. Ltd.).

### Results

Fluorescence was observed from all dots discharged from the thermal head. Fluorescence observed had an average intensity of 1600 under the conditions of a set sensitivity of HV=5.0 and the integration number of 64 for Argus 50. An average dot diameter was about 70 μm. A control experiment was conducted as described above substituting Compound VI with Compound IV which had the same basic structure as Compound VI and did not have a thiol group, giving an average fluorescence intensity of 130. Thus, it was confirmed that a fluorescent dye having a thiol group can be bound to the surface of a glass substrate.

### Example 3

Preparation of a marked DNA array substrate and hybridization

A marked DNA array substrate was prepared as described in Example 2. There will be described the base sequence of a DNA probe and a process for preparing the substrate.

FIG. 1 schematically shows the sequences of DNA probes on the DNA array and their arrangement. Specifically, for a single-stranded nucleic acid having SEQ ID NO. 1 as a target substance, there are disposed a probe having a completely complementary strand to the sequence of the target substance and probes with 1, 2 and 3 base mismatches to the sequence of the target substance, respectively.

SEQ ID NO. 1: ^{5′}ATGAACCGGAGGCCCATC^{3′}

In a probe spot at a certain number, there is a probe having a sequence of each SEQ ID NO. as shown in Table 1.

TABLE 1

Spot No. 1: SEQ ID NO. 2
Spot No. 2: SEQ ID NO. 3
Spot No. 3: SEQ ID NO. 4
Spot No. 4: SEQ ID NO. 5
Spot No. 5: SEQ ID NO. 6
Spot No. 6: SEQ ID NO. 7
Spot No. 7: SEQ ID NO. 8
Spot No. 8: SEQ ID NO. 9
Spot No. 9: SEQ ID NO. 10
Spot No. 10: SEQ ID NO. 11
Spot No. 11: SEQ ID NO. 12
Spot No. 12: SEQ ID NO. 13
Spot No. 13: SEQ ID NO. 14
Spot No. 14: SEQ ID NO. 15
Spot No. 15: SEQ ID NO. 16
Spot No. 16: SEQ ID NO. 17
Spot No. 17: SEQ ID NO. 18
Spot No. 18: SEQ ID NO. 19
Spot No. 19: SEQ ID NO. 20
Spot No. 20: SEQ ID NO. 21
Spot No. 21: SEQ ID NO. 22
Spot No. 22: SEQ ID NO. 23
Spot No. 23: SEQ ID NO. 24
Spot No. 24: SEQ ID NO. 25
Spot No. 25: SEQ ID NO. 26
Spot No. 26: SEQ ID NO. 27
Spot No. 27: SEQ ID NO. 28
Spot No. 28: SEQ ID NO. 29
Spot No. 29: SEQ ID NO. 30
Spot No. 30: SEQ ID NO. 31
Spot No. 31: SEQ ID NO. 32
Spot No. 32: SEQ ID NO. 33
Spot No. 33: SEQ ID NO. 34
Spot No. 34: SEQ ID NO. 35
Spot No. 35: SEQ ID NO. 36
Spot No. 36: SEQ ID NO. 37
Spot No. 37: SEQ ID NO. 38
Spot No. 38: SEQ ID NO. 39
Spot No. 39: SEQ ID NO. 40
Spot No. 40: SEQ ID NO. 41
Spot No. 41: SEQ ID NO. 42
Spot No. 42: SEQ ID NO. 43
Spot No. 43: SEQ ID NO. 44
Spot No. 44: SEQ ID NO. 45
Spot No. 45: SEQ ID NO. 46
Spot No. 46: SEQ ID NO. 47
Spot No. 47: SEQ ID NO. 48
Spot No. 48: SEQ ID NO. 49
Spot No. 49: SEQ ID NO. 50
Spot No. 50: SEQ ID NO. 51
Spot No. 51: SEQ ID NO. 52
Spot No. 52: SEQ ID NO. 53
Spot No. 53: SEQ ID NO. 54
Spot No. 54: SEQ ID NO. 55
Spot No. 55: SEQ ID NO. 56
Spot No. 56: SEQ ID NO. 57
Spot No. 57: SEQ ID NO. 58
Spot No. 58: SEQ ID NO. 59
Spot No. 59: SEQ ID NO. 60
Spot No. 60: SEQ ID NO. 61
Spot No. 61: SEQ ID NO. 62
Spot No. 62: SEQ ID NO. 63
Spot No. 63: SEQ ID NO. 64
Spot No. 64: SEQ ID NO. 65
—
—
—
—
—
—
—
—
—
—
—

SEQ ID NO. 1 is complementary to the sequence of the target DNA while the other sequences are complementary to SEQ ID NO. 1. In terms of three bases underlined in each SEQ ID NO., there are all combinations of A, G, C and T, i.e., 43=64 sequences. Three bases in the above probe correspond to those underlined in SEQ ID NO. 1, respectively. “N” in a sequence in each probe array shown in FIG. 1 corresponds to A, G, C or T as indicated outside of the upper side in each probe array. As a result, among the spots on the substrate shown in FIG. 1, No. 42 corresponds to a completely complementary probe to the target DNA sequence; Nos. 10, 26, 34, 38, 41, 43, 44, 46 and 58 correspond to probes with one base mismatch to the target DNA sequence; Nos. 2, 6, 9, 11, 12, 14, 18, 22, 25, 27, 28, 30, 33, 35, 36, 37, 39, 40, 45, 47, 48, 50, 54, 57, 59, 60 and 62 correspond to probes with two base mismatches to the target DNA sequence; and the others correspond to probes with three base mismatches to the target DNA sequence.

All of these 65 DNA including a rhodamine labeling model target DNA were purchased from Becks Inc. A probe DNA had a thiol linker at its 5-terminal for attachment to the substrate. An example of a DNA having a thiol linker is Compound VIII below. Compound VIII has a completely complementary sequence (No. 42) to the model target DNA.

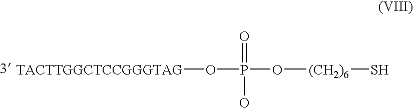

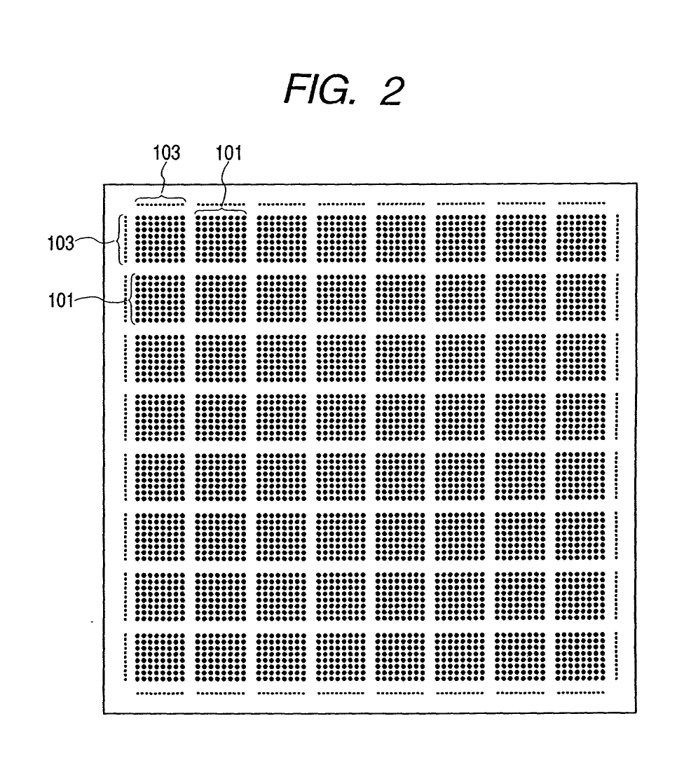

These 64 DNA probes and Compound VI were discharged for reaction on a glass substrate as described in Example 2. A concentration during discharging a DNA probe was 1.5 OD/2 mL. In this example, a spot of one DNA probe was practically formed from 8×8=64 dots and dots 101 (8 per 1 position×32 positions) of Compound VI were disposed around the periphery of the square formed by the 64 DNA probes (See FIG. 2). Factors such as a dot diameter and a pitch were as in Example 2. The substrate was washed as described in Example 2, subject to blocking with BSA for preventing non-specific adsorption of, e.g., DNA on the surface, washed with a phosphate buffer used in hybridization (10 mM phosphate buffer, pH=7.0, containing 5 mM NaCl) and then subject to hybridization.

Hybridization was conducted in a hybripack using 2 ml of the above buffer containing the target DNA (No. 65) at 5 nM. The substrate was placed in the hybripack together with the target DNA solution. The pack was sealed, heated to 75° C. in an incubator, cooled to 45° C. and then maintained under the conditions for 10 hours.

Then, the substrate was removed from the

Then, the substrate was removed from the hybripack, washed with the buffer for hybridization and observed for fluorescence as described in Example 2.

### Results

Fluorescence was observed from all the dots containing Compound VI on the substrate. Fluorescence observed had an average intensity of 3900 under the conditions as described in Example 2 except a set sensitivity of HV=2.0 for Argus 50. For the dots of the DNA probes, fluorescence was observed from 64 dots formed by one DNA probe and the position was specified to be of the DNA probe No. 42 from the dots of Compound VI. An average fluorescence intensity was 1800 (a set sensitivity of HV=5.0 for Argus 50). These results indicate that a marking method of this invention is effective for detecting and quantifying a target DNA using a DNA probe array and that since information such as a fluorescence intensity obtained from a marking position provided by the marking method of this invention are substantially constant if the conditions such as a device are constant, it may be used as a standard signal quantity to correct a signal quantity from a sample.

This invention allows a solid substrate to be marked. The marking method of this invention may be employed to conveniently and reliably detect a target substance using a solid probe array.

What is claimed is:

1. A probe bound substrate on which a probe capable of specifically attaching to a target substance is bound at a first site on a surface of the substrate, characterized in that a marker is bound at a second site where the first site can be specified.

2. The probe bound substrate according to claim 1 wherein said marker is a dye.

3. The probe bound substrate according to claim 1 wherein said marker is a fluorescent material.

4. The probe bound substrate according to claim 2 wherein said marker is a fluorescent dye.

5. The probe bound substrate according to claim 1 wherein said probe is a single-stranded nucleic acid.

6. The probe bound substrate according to claim 5 wherein said probe is a single-stranded DNA.

7. The probe bound substrate according to claim 5 wherein said probe is a single-stranded RNA.

8. The probe bound substrate according to claim 5 wherein said probe is a single-stranded PNA (peptide nucleic acid).

9. A process for manufacturing a probe bound substrate comprising the steps of: applying a solution containing a probe capable of specifically making a bond with a target substance and having a second functional group capable of making a bond with a first functional group attached to the surface of a substrate, to a first site of a surface of a substrate and binding the probe to the substrate at the first site of a substrate surface, further comprising the step of:
applying a solution containing a marker having a third functional group capable of directly or indirectly making a bond with said first functional group to a second site of the substrate surface binding said maker to the second position of said substrate surface and wherein said first site can be specified from said second site.

10. The process according to claim 9 wherein said marker is a dye.

11. The process according to claim 9 wherein said marker is a fluorescent material.

12. The process according to claim 10 wherein said marker is a fluorescent dye.

13. The process according to claim 9 wherein said first functional group is maleimide and said third functional group is thiol.

14. The process according to claim 9 wherein said first functional group is thiol and said third functional group is maleimide.

15. The process according to claim 9 wherein said first functional group is succinimide and said third functional group is amino.

16. The process according to claim 9 wherein said first functional group is amino and said third functional group is succinimide.

17. The process according to claim 9 wherein said first functional group is isocyanate and said third functional group is amino.

18. The process according to claim 9 wherein said first functional group is amino and said third functional group is isocyanate.

19. The process according to claim 9 wherein said first functional group is chloride and said third functional group is hydroxyl.

20. The process according to claim 9 wherein said first functional group is epoxy and said third functional group is amino.

21. The process according to claim 9 wherein said first functional group is carboxy and said third functional group is hydroxyl.

22. The process according to claim 9 wherein said first functional group is hydroxyl and said third functional group is carboxy.

23. The process according to claim 9 wherein said probe is a single-stranded nucleic acid.

24. The process according to claim 23 wherein said probe is a single-stranded DNA.

25. The process according to claim 23 wherein said probe is a single-stranded RNA.

26. The process according to claim 23 wherein said probe is a single-stranded PNA (peptide nucleic acid).

27. The process according to claim 9 wherein said substrate is a glass substrate.

28. The process according to claim 27 wherein said substrate is a glass substrate to which a silane coupling agent having said first functional group at one end is attached at its other end.

29. The process according to claim 28 wherein said first functional group is thiol.

30. The process according to claim 28 wherein said first functional group is amino.

31. The process according to claim 28 wherein said first functional group is isocyanate.

32. The process according to claim 28 wherein said first functional group is chloride.

33. The process according to claim 28 wherein said first functional group is epoxy.

34. The process according to claim 27 wherein said substrate is a glass substrate to which a silane coupling agent having said first functional group at one end is attached at its other end; and the maker is bound to the surface of the substrate via a linker having a fourth functional group capable of making a bond with said first functional group at one end and a fifth functional group capable of making a bond with the third functional group at the other end.

35. The process according to claim 34 wherein said first, said fourth and said fifth functional groups are amino, succinimide and maleimide, respectively and said third functional group is thiol.

36. The process according to claim 35 wherein said thiol group as the third functional group is introduced into the marker by binding N-succinimidyl-3-(2-pyridyldithio)propionate to an amino group in a precursor of the marker and then converting it into a thiol group by cleaving a disulfide (-SS-) moiety formed.

37. The process according to claim 34 wherein said first, said fourth and said fifth functional groups are thiol, maleimide and succinimide, respectively and said third functional group is amino.

38. The process according to claim 34 wherein the linker is N-(6-maleimidocaproxy)succinimide.

39. The process according to claim 34 comprising the steps of applying said linker to the second position to which said marker is to be applied, on the substrate having said first functional group at one end and applying said marker to the position in which said linker has been applied.

40. The process according to claim 9 or 39 wherein application of said marker to the surface of the substrate is performed by discharging a liquid containing said marker by ink jet technique.

41. The process according to claim 40 wherein said ink jet technique is thermal jet technique.

42. The process according to claim 40 wherein said ink jet technique is piezo jet technique.

43. The process according to claim 40 wherein the liquid containing said marker contains 5 to 10 wt % of urea, 5 to 10 wt % of glycerol, 5 to 10 wt % of thiodiglycol and 1 wt % of an acetylene alcohol to the whole amount of the liquid.

44. The process according to claim 43 wherein the acetylene alcohol has the structure represented by general formula I: 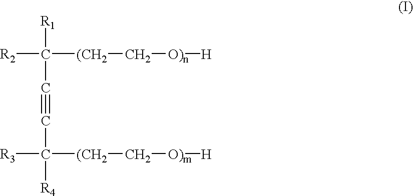
wherein R1, R2, R3 and R4 independently represent alkyl; m and n independently represent an integer provided that m or n is zero when m=n=0 or 1≦m+n≦30 and m+n=1.

45. The process according to claim 9 wherein said first functional group is maleimide and said second functional group is thiol.

46. The process according to claim 9 wherein said first functional group is epoxy and said second functional group is amino.

47. The process according to claim 9 wherein application of the liquid containing said probe to the surface of the substrate is performed by discharging the liquid containing said probe by ink jet technique.

48. The process according to claim 47 wherein said ink jet technique is thermal jet technique.

49. The process according to claim 47 wherein said ink jet technique is piezo jet technique.

50. The process according to claim 47 wherein the liquid containing the probe contains 5 to 10 wt % of urea, 5 to 10 wt % of glycerol, 5 to 10 wt % of thiodiglycol and 1 wt % of an acetylene alcohol to the whole amount of the liquid.

51. The process according to claim 50 wherein the acetylene alcohol has the structure represented by general formula I: 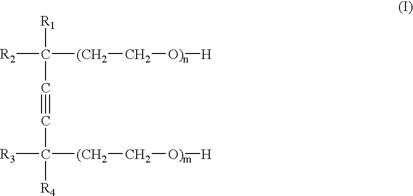
wherein R1, R2, R3 and R4 independently represent alkyl; m and n independently represent an integer provided that m or n is zero when m=n=0 or 1≦m+n≦30 and m+n=1.

52. A probe array comprising spots for mutually independent probes at multiple sites on a substrate surface wherein a marker is present on the substrate surface such that the positions of said spots can be specified.

53. The probe array according to claim 52 wherein said marker is a dye.

54. The probe array according to claim 52 wherein said marker is a fluorescent material.

55. The probe array according to claim 53 wherein said marker is a fluorescent dye.

56. The probe array according to claim 52 wherein said spots are disposed as a matrix and said marker is applied to a position which may be specified by a row and a column in the matrix.

57. A method of detecting a target substance comprising the steps of: contacting a sample with each spot in a probe array on a substrate, having probes capable of specifically making a bond with a target substance possibly contained in said sample as a plurality of mutually independent sopts, wherein a marker is present on a substrate surface such that the positions of said spots can be specified, and detecting the presence of a reaction product of said probe with said target substance in any spot to detect the presence of said target substance in said sample, further comprising the step of specifying the positions of said spots where said reaction product is present on the basis of the positions of the marker on said substrate surface when the presence of said reaction product is detected.

58. A method of sequencing a single-stranded nucleic acid in a sample comprising the steps of:
contacting said sample with each spot in a probe array having probes having a complementary sequence to each of expected multiple sequences in said single-stranded nucleic acid as a plurality of mutually independent spots, wherein and where a marker is present on a substrate surface such that the positions of the spots can be specified, and specifying the positions of said spots where a reaction product of said probe with a target substance has been formed on the basis of the positions of said marker on said substrate.

59. A method of quantifying a target substance wherein the quantity of fluorescence generated from a marker is used as a standard fluorescence quantity in a procedure where the probe array according to claim 53 is used for detecting and quantifying a target substance capable of specifically making a bond with probes by a fluorescent technique.


SEQUENCE LISTING (65 sequences)

SEQ ID NO: 1 - DNA, 18 bp, Artificial Sequence
  Probe Sequence
atgaaccggaggcccatc

SEQ ID NO: 2 - DNA, 18 bp, Artificial Sequence
  Probe Sequence
gatgggactcaagttcat

SEQ ID NO: 3 - DNA, 18 bp, Artificial Sequence
  Probe Sequence
gatgggactcaggttcat

SEQ ID NO: 4 - DNA, 18 bp, Artificial Sequence
  Probe Sequence
gatgggactcacgttcat

SEQ ID NO: 5 - DNA, 18 bp, Artificial Sequence
  Probe Sequence
gatgggactcatgttcat

SEQ ID NO: 6 - DNA, 18 bp, Artificial Sequence
  Probe Sequence
gatgggactcgagttcat

SEQ ID NO: 7 - DNA, 18 bp, Artificial Sequence
  Probe Sequence
gatgggactcgggttcat

SEQ ID NO: 8 - DNA, 18 bp, Artificial Sequence
  Probe Sequence
gatgggactcgcgttcat

SEQ ID NO: 9 - DNA, 18 bp, Artificial Sequence
  Probe Sequence
gatgggactcgtgttcat

SEQ ID NO: 10 - DNA, 18 bp, Artificial Sequence
  Probe Sequence
gatgggactccagttcat

SEQ ID NO: 11 - DNA, 18 bp, Artificial Sequence
  Probe Sequence
gatgggactccggttcat

SEQ ID NO: 12 - DNA, 18 bp, Artificial Sequence
  Probe Sequence
gatgggactcccgttcat

SEQ ID NO: 13 - DNA, 18 bp, Artificial Sequence
  Probe Sequence
gatgggactcctgttcat

SEQ ID NO: 14 - DNA, 18 bp, Artificial Sequence
  Probe Sequence
gatgggactctagttcat

SEQ ID NO: 15 - DNA, 18 bp, Artificial Sequence
  Probe Sequence
gatgggactctggttcat

SEQ ID NO: 16 - DNA, 18 bp, Artificial Sequence
  Probe Sequence
gatgggactctcgttcat

SEQ ID NO: 17 - DNA, 18 bp, Artificial Sequence
  Probe Sequence
gatgggactcttgttcat

SEQ ID NO: 18 - DNA, 18 bp, Artificial Sequence
  Probe Sequence
gatggggctcaagttcat

SEQ ID NO: 19 - DNA, 18 bp, Artificial Sequence
  Probe Sequence
gatggggctcaggttcat

SEQ ID NO: 20 - DNA, 18 bp, Artificial Sequence
  Probe Sequence
gatggggctcacgttcat

SEQ ID NO: 21 - DNA, 18 bp, Artificial Sequence
  Probe Sequence
gatggggctcatgttcat

SEQ ID NO: 22 - DNA, 18 bp, Artificial Sequence
  Probe Sequence
gatggggctcgagttcat

SEQ ID NO: 23 - DNA, 18 bp, Artificial Sequence
  Probe Sequence
gatggggctcgggttcat

SEQ ID NO: 24 - DNA, 18 bp, Artificial Sequence
  Probe Sequence
gatggggctcgcgttcat

SEQ ID NO: 25 - DNA, 18 bp, Artificial Sequence
  Probe Sequence
gatggggctcgtgttcat

SEQ ID NO: 26 - DNA, 18 bp, Artificial Sequence
  Probe Sequence
gatggggctccagttcat

SEQ ID NO: 27 - DNA, 18 bp, Artificial Sequence
  Probe Sequence
gatggggctccggttcat

SEQ ID NO: 28 - DNA, 18 bp, Artificial Sequence
  Probe Sequence
gatggggctcccgttcat

SEQ ID NO: 29 - DNA, 18 bp, Artificial Sequence
  Probe Sequence
gatggggctcctgttcat

SEQ ID NO: 30 - DNA, 18 bp, Artificial Sequence
  Probe Sequence
gatggggctctagttcat

SEQ ID NO: 31 - DNA, 18 bp, Artificial Sequence
  Probe Sequence
gatggggctctggttcat

SEQ ID NO: 32 - DNA, 18 bp, Artificial Sequence
  Probe Sequence
gatggggctctcgttcat

SEQ ID NO: 33 - DNA, 18 bp, Artificial Sequence
  Probe Sequence
gatggggctcttgttcat

SEQ ID NO: 34 - DNA, 18 bp, Artificial Sequence
  Probe Sequence
gatgggcctcaagttcat

SEQ ID NO: 35 - DNA, 18 bp, Artificial Sequence
  Probe Sequence
gatgggcctcaggttcat

SEQ ID NO: 36 - DNA, 18 bp, Artificial Sequence
  Probe Sequence
gatgggcctcacgttcat

SEQ ID NO: 37 - DNA, 18 bp, Artificial Sequence
  Probe Sequence
gatgggcctcatgttcat

SEQ ID NO: 38 - DNA, 18 bp, Artificial Sequence
  Probe Sequence
gatgggcctcgagttcat

SEQ ID NO: 39 - DNA, 18 bp, Artificial Sequence
  Probe Sequence
gatgggcctcgggttcat

SEQ ID NO: 40 - DNA, 18 bp, Artificial Sequence
  Probe Sequence
gatgggcctcgcgttcat

SEQ ID NO: 41 - DNA, 18 bp, Artificial Sequence
  Probe Sequence
gatgggcctcgtgttcat

SEQ ID NO: 42 - DNA, 18 bp, Artificial Sequence
  Probe Sequence
gatgggcctccagttcat

SEQ ID NO: 43 - DNA, 18 bp, Artificial Sequence
  Probe Sequence
gatgggcctccggttcat

SEQ ID NO: 44 - DNA, 18 bp, Artificial Sequence
  Probe Sequence
gatgggcctcccgttcat

SEQ ID NO: 45 - DNA, 18 bp, Artificial sequence
  Probe Sequence
gatgggcctcctgttcat

SEQ ID NO: 46 - DNA, 18 bp, Artificial Sequence
  Probe Sequence
gatgggcctctagttcat

SEQ ID NO: 47 - DNA, 18 bp, Artificial Sequence
  Probe Sequence
gatgggcctctggttcat

SEQ ID NO: 48 - DNA, 18 bp, Artificial Sequence
  Probe Sequence
gatgggcctctcgttcat

SEQ ID NO: 49 - DNA, 18 bp, Artificial Sequence
  Probe Sequence
gatgggcctcttgttcat

SEQ ID NO: 50 - DNA, 18 bp, Artificial Sequence
  Probe Sequence
gatgggtctcaagttcat

SEQ ID NO: 51 - DNA, 18 bp, Artificial Sequence
  Probe Sequence
gatgggtctcaggttcat

SEQ ID NO: 52 - DNA, 18 bp, Artificial Sequence
  Probe Sequence
gatgggtctcacgttcat

SEQ ID NO: 53 - DNA, 18 bp, Artificial Sequence
  Probe Sequence
gatgggtctcatgttcat

SEQ ID NO: 54 - DNA, 18 bp, Artificial Sequence
  Probe Sequence
gatgggtctcgagttcat

SEQ ID NO: 55 - DNA, 18 bp, Artificial Sequence
  Probe Sequence
gatgggtctcgggttcat

SEQ ID NO: 56 - DNA, 18 bp, Artificial Sequence
  Probe Sequence
gatgggtctcgcgttcat

SEQ ID NO: 57 - DNA, 18 bp, Artificial Sequence
  Probe Sequence
gatgggtctcgtgttcat

SEQ ID NO: 58 - DNA, 18 bp, Artificial Sequence
  Probe Sequence
gatgggtctccagttcat

SEQ ID NO: 59 - DNA, 18 bp, Artificial Sequence
  Probe Sequence
gatgggtctccggttcat

SEQ ID NO: 60 - DNA, 18 bp, Artificial Sequence
  Probe Sequence
gatgggtctcccgttcat

SEQ ID NO: 61 - DNA, 18 bp, Artificial Sequence
  Probe Sequence
gatgggtctcctgttcat

SEQ ID NO: 62 - DNA, 18 bp, Artificial Sequence
  Probe Sequence
gatgggtctctagttcat

SEQ ID NO: 63 - DNA, 18 bp, Artificial Sequence
  Probe Sequence
gatgggtctctggttcat

SEQ ID NO: 64 - DNA, 18 bp, Artificial Sequence
  Probe Sequence
gatgggtctctcgttcat

SEQ ID NO: 65 - DNA, 18 bp, Artificial Sequence
  Probe Sequence
gatgggtctcttgttcat